(19) United States
(12) Patent Application Publication
(10) Pub. No.: US 2006/0289020 A1
(43) Pub. Date: Dec. 28, 2006

(54) System and method for dynamic determination of disease prognosis

(76) Inventors: Ying P. Tabak, Weston, MA (US); Richard S. Johannes, Newton, MA (US); Stephen G. Kurtz, Sudbury, MA (US); Cynthia Yamaga, Oceanside, CA (US)

Correspondence Address: FULWIDER PATTON, 6060 CENTER DRIVE, 10TH FLOOR, LOS ANGELES, CA, 90045, US

(21) Appl. No.: 11/449,450

(22) Filed: Jun. 8, 2006

Related U.S. Application Data
(60) Provisional application No. 60/688,858, filed on Jun. 8, 2005.

(51) Int. Cl. G06F 19/00; A61B 19/00

(52) U.S. Cl. 128/898; 702/19

(57) ABSTRACT

A method of obtaining and processing patient data and patient treatment data to provide a prognosis parameter related to a patient's disease state is provided. The method identifies and calculates coefficients related to appropriate predictor variables which are then used by the prediction model to calculate the prognosis parameter. The prediction model may be a logistic regression model. The method may also be used to assess the level of care being provided to patients, as well as providing a way of assessing the outcome of the patient's condition as a function of treatment. A method of calculating a harm index reflective of the risk of treatment is also provided.

### RELATED APPLICATIONS

This application is based on, and claims the benefit of priority to, U.S. Provisional Application Ser. No. 60/688,858, filed Jun. 8, 2005, which is incorporated by reference herein in its entirety.

### FIELD OF THE INVENTION

The invention generally relates to a medical decision support system and more specifically for the dynamically determining a prognosis of a medical disorder for a patient.

### BACKGROUND OF THE INVENTION

As used herein, the term “disease” is defined as a deviation from the normal structure or function of any part, organ or system of the body (or any combination thereof). A specific disease is manifested by characteristic symptoms and signs, including both chemical and physical changes. A disease is often associated with a variety of other factors including but not limited to demographic, environmental, employment, genetic and medically historical factors. Certain characteristic signs, symptoms, and related factors can be quantitated through a variety of methods to yield important diagnostic information. Current diagnostic and prognostic methods depend on the identification and evaluation of variables, or markers associated with a given disease state, both individually and as they relate to one another. Often the diagnosis of a particular disease involves the subjective analysis by a clinician, such as a physician, veterinarian, or other health care provider, of the data obtained from the measurement of the factors mentioned above in conjunction with a consideration of many of the traditionally less quantitative factors such as employment history. Unfortunately, this subjective process of diagnosing or prognosing a disease usually cannot accommodate all potentially relevant factors and provide an accurate weighting of their contribution to a correct diagnosis or prognosis.

Generally, the pathological process involves gradual changes that become apparent only when overt change has occurred. In many instances, pathological changes involve subtle alterations in multiple variables or markers. It is uncommon that a single marker will be indicative of the presence or absence of a disease. It is the pattern of those markers relative to one another and relative to a normal reference range, that is indicative of the presence of a disease. Additional factors including but not limited to demographic, environmental, employment, genetic and medically historical factors may contribute significantly to the diagnosis or prognosis of a disease, especially when considered in conjunction with patterns of markers. Unfortunately, the subjective diagnostic process of considering the multiple factors associated with the cause or presence of a disease is somewhat imprecise and many factors that may contribute significantly are not afforded sufficient weight or considered at all.

When individual markers do not show a predictable change and the patterns and interrelationships among the markers viewed collectively are not clear, the accuracy of a physician's diagnosis is significantly reduced. Also, as the number of markers and demographic variables relevant for the diagnosis of a particular disease increases, the number of relevant diagnostic patterns among these variables increases. This increasing complexity decreases the clinician's ability to recognize patterns and accurately diagnose or predict disease prognosis.

Various attempts have been made to develop models to assess and analyze databases in a retrospective fashion that are capable of predicting an expected morbidity of a patient presenting for treatment at an institution. In one example, longitudinal data is extracted from a database containing longitudinal data for a plurality of patients, and predictive modeling techniques are then used to predict a clinical outcome for a patient.

In another system, a retrospective cohort study was carried out on thousands of intensive care unit admissions to quantify the variability in risk-adjusted mortality and length of stay in intensive care units using a computer-based severity of illness measure. One disadvantage of each of the prior methods is that each focuses retrospectively, and does not attempt to use the wealth of stored data to be found within an institution's data bases to provide a quantification of the probability of improvement or to identify when a patients status is declining, or where the length of stay of the patient is beyond a predetermined range indicative of successful treatment.

What has been needed, and heretofore unavailable, is a system and method that allows application of population-based predicative models in real time. Such a system and method would provide for improved clinical care and outcomes by identifying outliers in real time, that is, for example, identifying patients who are not responding as expected within a specified time frame. Moreover, such a system should be automated so that it can communicate with other institutional systems so as to provide an alarm when the real time prediction of the prognosis of the patient exceeds an institutionally established guideline. Additionally, such a system will also result in improved resource management of the institution by predicting the acuity of patients disease states and providing input for ensuring that the proper staff are on call at appropriate levels to be able to deliver the amount of care necessary to adequately care for the institution's patients. Thus the system and method should be capable of identifying mismatches in level of care and patient disease acuity, providing an early warning for patients whose clinical condition is deteriorating, or signaling to check on those patients who may be able to be moved to a lower level of care or discharge.

Further, there is a need for a system that simultaneously evaluates and quantifies risk for treatment of a patient, assisting in identifying the optimal treatment to be given to a patient in a predictive, predicable manner based on best practices derived in an empirical manner from the data stored in an institution's databases. Such a system would allow use of automated data analysis to provide a real time severity of illness scoring that may be used as a cost-effective monitoring tool. Moreover, continuous analysis of real time data gathered on current patients allows for improving the model based on retrospective analysis of the institution's databases, and improving the predictability of the system as the system learns from the current patient treatments and the patients' response to those treatments. The present invention satisfies these, and other needs.

### SUMMARY OF THE INVENTION

Briefly, and in general terms, in one aspect, the present invention includes a system and method for automatically extracting data from an institution's database or databases, calculating coefficients for appropriate predictor variables, and then incorporating current information from a patient to determine a real time acuity/severity score, or other predictive value, that may be used to assess a patient's condition, to assist in determining an appropriate course of treatment, and to monitor the progress of the patient. In another aspect, the present invention provides a system and method for alerting caregivers when a patient's course of treatment needs to be reassessed or changed, or when the level of care being provided to the patient needs modification.

In another aspect, the system and method of the present invention provides a tool for assessing and monitoring resource management of an institution by providing for prediction of acuity of patients and flexing the staff of the institution by function level and experience or expertise. Moreover, in other aspects, the present invention provides for identifying miss-matches in level of care and patient acuity, thus providing an early warning for patients whose clinical condition is deteriorating, or who may be able to be moved to a lower level of care.

In a further aspect, the present invention incorporates a real time data feed that allows the predictive model to be continuously improved. In this manner, the predictive power of the model increases as more data related to patient treatment and patient response to that treatment is acquired.

In still another aspect, the acuity/severity score or other prediction value is communicated to a harm index engine and incorporated into the calculation of a medication harm index that is used to quantify the risk of a particular course of treatment.

Other features and advantages of the invention will become apparent from the following detailed description, taken in conjunction with the accompanying drawings, which illustrate, by way of example, the features of the invention

### BRIEF DESCRIPTION OF THE DRAWINGS

FIG. 1 is a schematic diagram of a institution-wide information and therapy management system incorporating principles of the present invention;

FIG. 2 is a schematic diagram showing details of elements of the institution-wide information and therapy management system of FIG. 1;

FIG. 3 is a schematic diagram showing details of the application of an acuity/severity score determined in accordance with the principles of the present invention to determining and monitoring treatment of a patient in an institution.

### DETAILED DESCRIPTION OF THE INVENTION

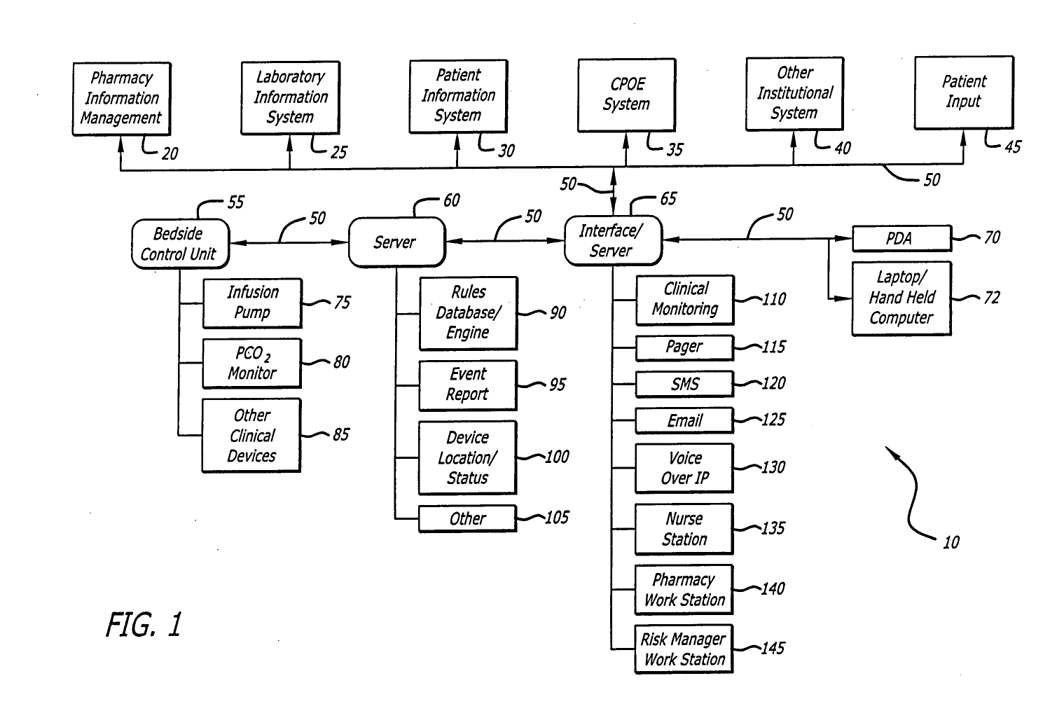

Referring now to drawings in which like reference numerals are used to refer to like or corresponding elements among the figures, there is generally shown in FIG. 1 an integrated hospital-wide information and therapy management system 10 in accordance with aspects of the present invention. The exemplary system depicted in FIG. 1 shows various institutional information systems, such as a pharmacy information management system 20, a laboratory information system 25, a patient information system 30, a computerized order entry system 35, a patient input system 45 and may include other institutional systems, such as other institutional system 40, as well. These systems are connected together using a suitable communications system 50, which includes various hardware, such as servers, routers, hard wire communication lines, and/or wireless network gear, such as wireless transmitters/receivers, routers, concentrators and the like. It will be immediately clear to those skilled in the art that such systems include processors and memory and are programmable and function under the control and operation of suitable software programs that may be embedded in various hardware devices, stored as programs in server memory or otherwise made available when needed and called for by the requirements of the systems.

The communications system 50 also connects the institutional systems described above with various systems that administer and monitor delivery of medical therapy to patients in the care giving institution. For example, there may be a bedside control or management unit 55 located in the general location of one or more patients, such as at a patient's bedside. The bedside controller 55 may be a dedicated device having a processor and memory and communication capability, and the processor is typically configured to run suitable software programs that may be stored in controller memory or downloaded over communication system 50 that allow the controller 55 to receive and transmit information and device operating instructions or receive patient treatment parameters to program and operate a variety of clinical devices that are controlled by the controller 55.

The controller 55 may also monitor the progress of treatment, including the start of treatment administered to the patient and alarms or changes to the treatment plan occurring during treatment, and also provide information about the course of treatment back to the system so that such information may be communicated to appropriate personnel or institutional systems. The bedside controller 55 may also take the form of a portable computing device or terminal that is in communication with the institution's network. The communication connection may be wired or wireless.

Various devices may be in communication with controller 55, and which may control their operation and also collect data for communication to other systems or it may control the communication of data from a device to other systems. For example only, and not limited to, controller 55 may control and monitor such devices as an infusion pump 75, PCO2 monitor 80 and other clinical devices such as a breathing rate sensor, pulse rate sensor, body temperature sensor, blood pressure sensor, urinary discharge volume sensor, an EKG sensor module, an EEG sensor module, an oxygen analyzer, a fetal monitor, a respirator, or other devices for maintaining blood sugar, providing electric nerve stimulation, and providing physical therapy and the like.

Bedside controller 55 communicates with other institutional systems using communications system 50. In one embodiment, controller 55 sends information to and receives information and/or operational commands or parameters from server 60. Server 60 includes various modules such as a rules database and engine 90, event reporting module 95, a module for tracking clinical device location and status 100, and other modules 105, such as a reporting module that may generate either standardized reports for use within the institution, or which may be programmed by input from care givers, technicians, or other institutional personnel to provide customized reports.

As depicted in FIG. 1, server 60 may be a stand alone device, which may communicate over communication system 50 with other interfaces or servers, such as interface/server 65. Alternatively, interface/server 65 and server 60 may reside on the same physical device.

Interface/server 65 provides server services and interfaces for interfacing controller 55 and server 60 with other institutional information systems, such as the pharmacy information system 20, the laboratory information system 25, the patient (or hospital or clinical) information system 30, the computerized physician order entry system (CPOE) 35, the patient input system 45 and any other appropriate or available institutional systems 40. Additionally, interface/server 65 may include modules for monitoring clinical devices 110 connected to controller 55 or server 60, modules for sending alarms, alerts or other information to care giver personnel over a pager network 115, short message service (SMS) text messaging 120, email 125, voice over internet (VoIP) 130 and other modalities, such as a wireless personal digital assistant (PDA), wireless application protocol (WAP) enabled telephone and the like.

Interface/server 65 may provide status reports of administered therapy, allow input of information or modification of prescribed therapy regimes, and provide indications of alert or alarm conditions communicated by clinical devices in communication with controller 55 at nursing stations 135, a pharmacy work station 140, physician workstation and/or a risk management work station 145. Interface/server 65 may also communicate with remote equipment, such as a PDA 70, or a lap-top or hand held computer 72. Such mobile, remote equipment may be carried by care givers, or mounted on or other wise associated with mobile institutional equipment to allow access by care givers to institutional data bases, allow for providing or altering therapy regimens, and for providing alerts, alarms or desired reports to care givers as they move about the institution.

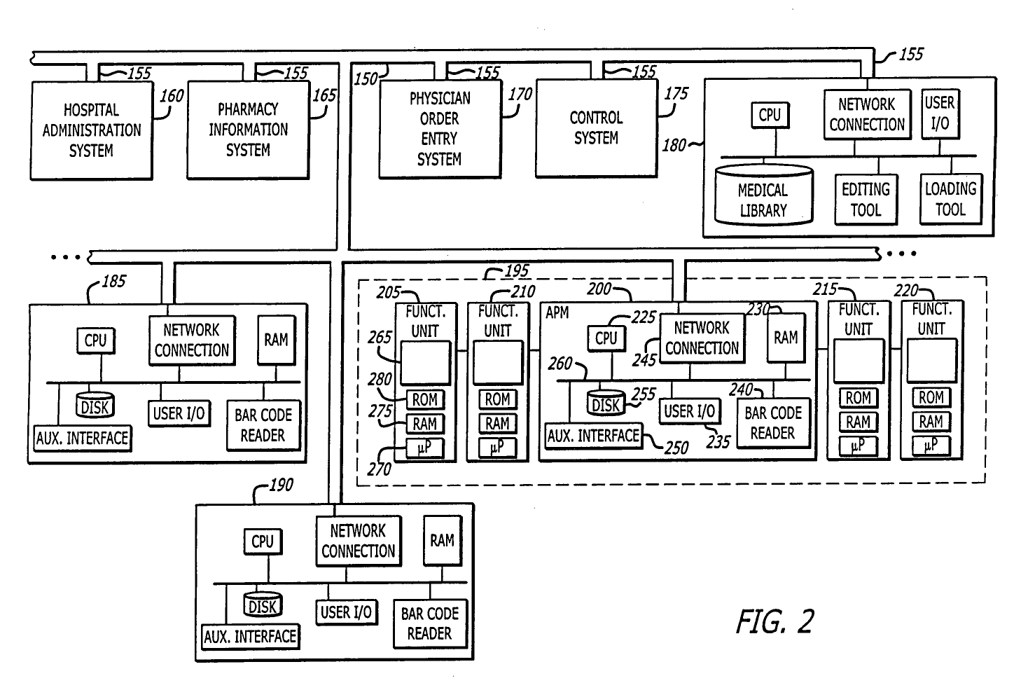

FIG. 2 depicts another example of a system incorporating aspects of the present invention and illustrating additional details of various components of the system. Various subsystems of the facility's information and therapy management system are connected together by way of a communication system 150. The communication system 150 may be, for example, a local area network (LAN), a wide area network (WAN), Inter- or intranet based, or some other communication network designed to carry signals allowing communications between the various information systems in the facility. For example, as shown in FIG. 2, the communication system 150 connects, through various interfaces 155, a hospital administration system 160, a pharmacy information system 165, a computerized physician order entry (CPOE) system 170, a control system 175, and a rules library 180. A plurality of patient care devices or systems 185, 190 and 195 may also be connected to communication system 150, either directly or through suitable routers, servers or other appropriate devices.

The communication system 150 may comprise, for example, an Ethernet (IEEE 522.3), a token ring network, or other suitable network topology, utilizing either wire or optical telecommunication cabling. In an alternative embodiment, the communication system 150 may comprise a wireless system, utilizing transmitters and receivers positioned throughout the care-giving facility and/or attached to various subsystems, computers, patient care devices and other equipment used in the facility. In such a wireless system, the signals transmitted and received by the system could be radio frequency (RF), infrared (IR), or other means capable of carrying information in a wireless manner between devices having appropriate transmitters or receivers. It will be immediately understood by those skilled in the art that such a system may be identical to the system set forth in FIGS. 1 and 2, with the exception that no wires are required to connect the various aspects of the system.

Each of the various systems 160, 165, 170, 175 and 180 generally comprise a combination of hardware such as digital computers which may include one or more central processing units, high speed instruction and data storage, on-line mass storage of operating software and short term storage of data, off-line long-term storage of data, such as removable disk drive platters, CD ROMs, or magnetic tape, and a variety of communication ports for connecting to modems, local or wide area networks, such as the network 150, and printers for generating reports. Such systems may also include remote terminals including video displays and keyboards, touch screens, printers and interfaces to a variety of clinical devices. The processors or CPUs of the various systems are typically controlled by a computer program or programs for carrying out various aspects of the present invention, as will be discussed more fully below, and basic operational software, such as a Windows™ operating system, such as Windows NT™, or Windows 2000™, or Windows XP™, distributed by Microsoft, Inc., or another operating program distributed, for example, by Linux, Red Hat, or any other suitable operating system. The operational software will also include various auxiliary programs enabling communications with other hardware or networks, data input and output and report generation and printing, among other functions.

While the system of the present invention is described with reference to various embodiments encompassing institutional wide information systems, those skilled in the art will recognize that the concepts and methodology of the present invention apply equally to information systems having a smaller scope. Embodiments of the system of the present invention can be designed to provide the functions and features of the present invention at the ward or department level. Such systems would include appropriate servers, databases, and communication means located within the ward to provide both wired and wireless connection between the various information systems, sensing devices and therapy delivery devices of the ward or department.

Patient care devices and systems 185, 190 and 195 may comprise a variety of diverse medical devices including therapeutic instruments such as parenteral and enteral infusion pumps and respirators, physiological monitors such as heart rate, blood pressure, ECG, EEG, and pulse oximeters, and clinical laboratory biochemistry instruments such as blood, urine and tissue sample measurement instruments and systems.

Additionally, the system may incorporate computerized inventory and distribution management appliances and systems. For example, the system may include drug distribution cabinets or controlled inventories that are located in areas of the institution other than the pharmacy. One example of such a system is described in U.S. Pat. No. 6,338,007, the subject matter of which is incorporated herein in its entirety.

It should be apparent to those skilled in the art that the systems described above can be simple or complex, depending on the needs of the institution. One advantage of such systems is that they provide a way to track the treatment being given to a patient, and through methods well known to those in the field, allow for the association of the treatment with various other patient information and physical parameters. Moreover, all of this information may be collated and analyzed in a real time fashion, allowing for the correlation of treatment to diagnostic tests, such as laboratory tests and monitored vital signs. This correlation, as will be discussed in more detail below, provides for real time determination of cause and effect. That is, it provides a care giver with feed back on the progress of the patient as a function of the treatment given.

In one embodiment, the present invention provides a method of applying population based predictive models in real time to the information that is being accumulated during the treatment of a patient. Moreover, this embodiment of the present invention provides a dynamic learning system that builds on the clinical outcomes of past patients, categorized by treatment type, disease type and status, and other variables, to provide a real time prognosis of how a patient should progress as treatment is administered. In the event that the patient's status does not change as expected, the system can provide an early warning to the caregiver that the treatment is not achieving the expected result, and, in some embodiments, may also provide advice based on rules and models incorporated in the software of the system to the caregiver to alter or enhance the patient's treatment.

As will be discussed in more detail below, various embodiments of the system and methods of the present invention provide information that is valuable as a resource management tool to assist an institution's management in ensuring that adequate levels of care are available to treat the number of patients in an institution, taking account of the severity of their illnesses and expected treatment course.

In an exemplary embodiment of the present invention, a logistic regression model is developed for a particular disease or condition, and then that model is used to determine a prognosis value for a current patient. Logistic regression analysis is a statistical method for determining the relationship between a dichotomous outcome variable and a set of predictor variables. It can be expressed as an equation:
Probability of outcome (e.g. death) =1/[1+e^{-(β}^{₀}^{+β}^{₁}^{χ}^{₁}^{+β}^{₂}^{χ}^{₂}^{+ . . . +β}^{ₙ}^{χ}^{ₙ}ⁿ)]

Where β₀ is the constant, Xᵢ's are predictor variables and βᵢ's are regression coefficients.

Each variable in the equation contains coefficients that play an important role in calculating the prediction. A coefficient can be either positive or negative, and are either discrete variables, such as those variables having yes or no answers, or continuous variables, where the variable value may be any value within a range of values. Generally speaking, a positive coefficient signifies an increased association with the outcome whereas a negative coefficient signifies a decreased association with the outcome. In other words, a positive coefficient in a mortality model indicates that the risk of mortality is higher in cases with this variable (discrete) or with higher values for the variable (continuous) than in cases without this variable (discrete) or that have lower values (continuous). As an example, a positive coefficient (yes) for cancer (discrete) would imply that cases with cancer have a higher risk of mortality than cases without cancer, all else being equal. A positive coefficient for age (continuous) would imply that patients with older age would have a higher risk of mortality than cases with younger age, all else being equal.

The coefficients in logistic regression can be interpreted as the log of the odds ratio (OR). Hence, the anti log of the coefficient is the OR for a one-unit increase in the variable or covariate. For example, the inventors have determined that the coefficient for age in the Ischemic Stroke mortality disease group is 0.038. It follows that OR_{1yr>75}=e⁰³⁸=1.04, meaning that each year increase of age after 75 is associated with a 4% increase in mortality, all else being equal.

As shown above, development of the model requires identification of variables to be used in the prediction model, as well as a determination of appropriate coefficients βᵢ. Typically, potential candidate variables are identified by reviewing the literature related to a desired disease or condition, the clinical relevance of the variable, and availability of the variable during the admission period of the patient. The variables are classified into demographics, laboratory findings (e.g. blood urea nitrogen, glucose), ICD-9 based principal diagnosis subcategories (e.g. staph aureus sepsis in septicemia, basal artery occlusion with infarction in ischemic stroke) and comorbidities (e.g. cancer, peripheral vascular disease), vital signs (systolic and diastolic blood pressure, temperature, respiration, and pulse) and altered mental status (level of consciousness).

Candidate variables associated with mortality at the univariate level (p<0.05) are then included as potential covariates in the multiple logistic regression model. Variable selection in multivariable modeling is also based on clinical and statistical significance. For each disease group the distribution and shape of continuous variables in the relationships with deaths is examined for each group. Continuous variables are crafted into multiple levels using recursive partitions, a statistical technique used to identify cut points to optimally differentiate multiple levels in a continuous distribution of a variable against the outcome.

To assess the incremental discriminatory power of each dimension of risk, demographics, laboratory findings, principal diagnosis subcategory, comorbidity, vital signs and altered mental status are entered into the multiple regression models sequentially. This order of blocked variables allows the prioritization of the contributions of objectively measured and automated lab data for ICD-9 based variables. Vital signs and altered mental status are modeled as the last block variables to assess the additional contribution of these currently manually collected data. The final predictive power of the model is then assessed by the area under the receiver operating characteristic (AUROC), a procedure well known to those skilled in the art.

Once the model is developed, it is validated internally using the bootstrap method by sampling with replacement for 200 iterations. A “bootstrap” algorithm draws random samples from the original database and fits a model on these samples, using the variables, which were selected in the stepwise algorithm. A model is fit on each sample, and variables that change sign between samples or are not found to be significant in seventy percent (70%) of the samples are dropped. The result is a final set of variables that are more robust and likely to behave the same way on a different set of data than the one used for initial variable selection.

The following example is useful in illustrating the above described method. An 85 year old patient is hospitalized with a principal diagnosis of ischemic stroke. At admission, the patient's creatinine level is greater than 3.0 mg./dL, glucose level is greater than 135 mg/dL. The patient has metastatic cancer, with a systolic blood pressure less than 90 mm Hg and a severely altered mental status.

Table 1 set forth below lists the coefficient estimates established for a variety of predictor variables. These coefficient estimates were calculated by analyzing data for 44,102 patients, of which 2929 died. The patient data used for these calculates is extracted from the database of the institution; the extraction may be done manually, which is time consuming and labor intensive, or the extraction is preferably done automatically, using data mining and analysis techniques well know to those skilled in the art.

TABLE 1
Ischemic Stroke Mortality Model

| | | | |
|---|---|---|---|
| | Co- | | |
| | efficient | | |
| Block | Estimate | P value | c-statistic |

2000-2001 Data 44102 Patients, 2929 Deaths

| | | |
|---|---|---|
| 0 | −4.20 | <.0001 |

Demographics

| | | | | |
|---|---|---|---|---|
| 1 | Yrs. >75 | 0.04 | <.0001 | 0.6049 |

Laboratory Findings

| | | | | |
|---|---|---|---|---|
| 2 | Albumin g/dL <= 2.7 | 0.27 | 0.0035 | 0.6225 |
| 2 | Creatinine >3.0 mg/dL | 0.82 | <.0001 | 0.6349 |
| 2 | Glucose >135 mg/dL | 0.33 | <.0001 | 0.6668 |
| 2 | pH Arterial <= 7.20 or >7.48 | 0.85 | <.0001 | 0.6910 |
| 2 | pH Arterial 7.21-7.35 | 0.61 | <.0001 | 0.7016 |
| 2 | WBC 10.9 k-14.1 k | 0.28 | <.0001 | 0.7070 |
| 2 | WBC >14.1 k | 0.58 | <.0001 | 0.7311 |
| 2 | PO2 <55/>140 or O2 <89/>98 | 0.67 | <.0001 | 0.7442 |
| 2 | PT INR >1.1 or PT/sec >13 | 0.35 | <.0001 | 0.7487 |

Principal Diagnosis and Comorbidities

| | | | | |
|---|---|---|---|---|
| 3 | Metastatic Cancer | 1.26 | <.0001 | 0.7528 |
| 3 | Basal Art Occl with Infarction | 1.30 | <.0001 | 0.7541 |

Vital Signs and Altered Mental Status (AMS)

| | | | | |
|---|---|---|---|---|
| 4 | Systolic BP <90 mm Hg | 0.70 | <.0001 | 0.7570 |
| 4 | Respirations <10 or >29/min | 0.67 | <.0001 | 0.7645 |
| 4 | Mild AMS | 0.83 | <.0001 | 0.7622 |
| 4 | Moderate AMS | 1.80 | <.0001 | 0.7674 |
| 4 | Severe AMS | 2.35 | <.0001 | 0.8298 |

Returning to the example, and using the coefficients set forth in Table 1, the probability of death of the patient may be calculated as follows:
Probability of death=1/[1+e^{-(-4.2+10(age >75)*0.04+1(creatinine)*0.82+1(glucose)*0.33 +1(metastasis)*1.26+1(SBP)*0.70+1(severe AMS) *2.35)}]=0.84

Thus, the patient of the example would have a predicted probability of death of 84%, a very severe case.

The system and method of the present invention is particularly advantageous in that it provides for tracking the progress of the patient and automatically updating the prognosis value with data that is collected concerning the patient's present condition. For example, as the patient of the above described example is treated, a body of data concerning her condition will be amassed in the database of the institution. For example, the database will acquire laboratory results, course of medication information, and information regarding physical examination and assessment by the patient's caregivers. This information is automatically input into the model to update the predicted probability of death. A change in the probability in one direction or the other indicates how the patient is responding to treatment, and may provide an early warning to care givers when the predicted probability of death is increasing, even in those cases where the trend is too subtle to be immediately discernible by caregivers.

The above example is just one possible use of the system and methods of the present invention, as those system and methods are applicable to not just a determination of the probability of death, but also have application to determining other aspects of the patient's progress, as well as being applicable to analyzing and assisting in resource management for the institution.

In various embodiments, the system and method provides for improved clinical care and outcomes by identifying outliers in real time, that is, for example, identifying patients who are not responding as expected within a specified time frame. For example, instead of calculating a prediction of a patient's probability of death, a model can be determined that predicts how long a patient is likely to remain hospitalized, based solely on the patient's condition at admission. Further, the system and method may be used to predict how long the patient will remain in a particular unit of the institution, such as ICU.

As set forth above., when the system is automated by incorporating appropriate software programs running on the institutions servers and other computers so it can communicate with other institutional systems, the system can provide an alarm when the real time prediction of the prognosis of the patient exceeds institutionally established guidelines that are contained in a database of rules. Additionally, such a system will also result in improved resource management of the institution by predicting the acuity of patients disease states and providing input for ensuring that the proper staff are on call at appropriate levels to be able to deliver the amount of care necessary to adequately care for the institutions patients. The system and method of various embodiments of the present invention are capable of identifying mismatches in level of care and patient disease acuity, providing an early warning for patients whose clinical condition is deteriorating, or signaling to check on those patients who may be able to be moved to a lower level of care.

By identifying appropriate predictor variables, the system simultaneously evaluates and quantifies risk for treatment of a patient, assisting in identifying the optimal treatment to be given to a patient in a predictive, predicable manner based on best practices derived in an empirical manner from the data stored in an institutions databases. Such a system allows use of automated data analysis to provide a real time severity of illness scoring that may be used as a cost-effective monitoring tool. Moreover, continuous analysis of real time data gathered on current patients allows for improving the model based on retrospective analysis of the institutions databases, improving the predictability of the system as the system learns from the current patient treatments and the patients' response to those treatments.

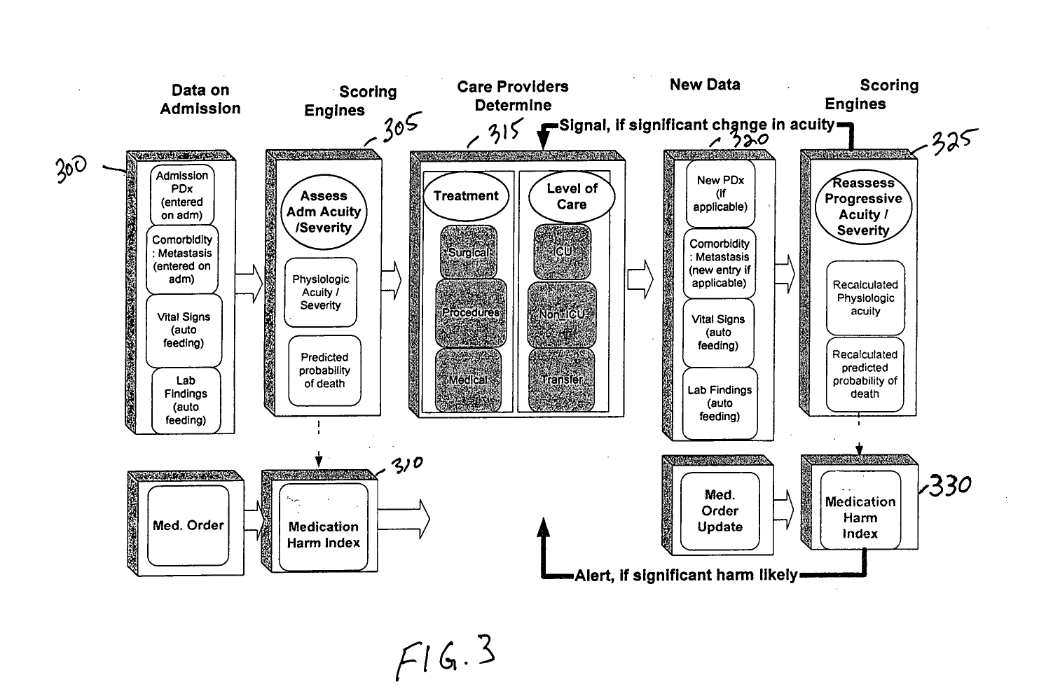

FIG. 3 provides a graphic illustration of the various embodiments of the system and methods of the present invention may be incorporated into the management of therapy provided to a patient in an institution. When a patient is admitted in box 300, four dimensions of data are collected and transmitted to the scoring engines utilizing the system and methods of the present invention embodied in software running on the institutions information management system. That data may be, for example, and not limited to, a principle diagnosis determined upon admission, any comorbidity data, such as the presence of metastasis, vital signs information, obtained either automatically or manually, and laboratory findings.

Once all of the above data is communicated to the scoring engines in box 305, the scoring engine generates an admission acuity/severity score, such as the predicted probability of death or other suitable score. The predicted acuity/severity score may then be used by caregivers in box 315 to determine the appropriate treatment and intensity of level of care needed, for example, ICU, non-ICU, or transfer to another ward, department or institution.

In box 320, the patient is treated, and during that treatment, additional, new and/or updated information related to the patients condition and status are gathered. For example, a new principle diagnosis may be made, additional vital signs data is accumulated and additional laboratory findings are acquired. All of this information is automatically fed back into the scoring engines in box 325, whereby the acuity/severity score is recalculated and updated. Depending on the results of this recalculation, the patient's treatment may be adjusted, or the level of intensity of care changed by caregivers; for example, the patient could be released from ICU into a non-ICU bed, or the opposite if warranted by the change in the patient's condition.

In another embodiment, the acuity/severity score may be further incorporated into determining a medication harm index calculation applied to a proposed treatment for a patient. For example, as shown in FIG. 3, the acuity/severity score calculated in box 305 may be automatically provided to a medication harm index engine 310 for incorporation into calculation of the harm index. Also, this harm index is updated in real time by automatically communicating any changes in the acuity/severity score, such as are calculated in box 325, into the harm index engine 330.

A harm index is a measure of harm that may occur to a patient if the patient is overdosed, or some other event, correlated with the course of treatment, occurs that is adverse to the patient. Various factors are considered in calculating a harm index. For example, factors may include such variables as detectability of an adverse event, the level of care being received by a patient, and the risk of a negative outcome given a certain dosage. These factors may be extracted by the system from the institution's database, and a single numeric index calculated using the methods describe above. In such a system, the higher the score, the greater risk or potential for harm to the patient.

In an automated system as described above, where medication administration to a patient may be monitored by one of the institution's devices, such as, for example, an infusion pump in communication with a bedside, or other, controller, the harm index associated with a given dosage being programmed into the device can be displayed to a user, or an alarm may be sounded to alert the user, so that the user may adjust the dosage. The same sort of method can be used where oral medication is being dispensed from a drug cabinet in communication with the institutions systems. In this example, if a medication is dispensed from a drug cabinet prior to order being entered into the system, a comparison to the calculated harm index may be made. If the harm index exceeds a predetermined level, the user may be alerted that the dose dispensed carries a risk of harm to the patient. This alert would allow the care giver to check the dosage before administering the medication to the patient.

While several particular forms of the invention have been illustrated and described, it will be apparent that various modifications can be made without departing from the spirit and scope of the invention.

We claim:

1. A method for determining a value for a prognosis parameter in real time, comprising:
obtaining current condition related information about a patient;
identifying appropriate predictor variables;
inputting the condition related information associated with the appropriate predictor variables into a prediction model;
calculating a value for a prognosis parameter.

2. The method of claim 1, wherein calculating a value for a prognosis parameter includes using calculated coefficients related to the predictor variables.

3. The method of claim 2, wherein the calculated coefficients are determined by analyzing a database of information containing condition related information obtained from a plurality of patients.

4. The method of claim 1, wherein the prediction model is a logistic regression model.

5. The method of claim 4, wherein the logistic regression determines a probability of outcome is equal to
1/[1+e^{-(β}^{₀}^{+β}^{₁}^{χ}^{₁}^{+β}^{₂}^{χ}^{₂}^{+ . . . +β}^{ₙ}^{χ}^{ₙ}⁾]
where β₀ is the constant, Xᵢ's are predictor variables and βᵢ's are regression coefficients.

6. The method of claim 2, wherein the coefficients related to the predictor values are continuously updated using individual patient information acquired during treatment of the patient.

7. The method of claim 6, further comprising monitoring a change in the value of the prognosis parameter.

8. The method of claim 7, further comprising adjusting the patient's treatment as a function of the monitored change in the value of the prognosis parameter.

9. The method of claim 7, further comprising adjusting a level of care provided to the patient as a function of the monitored change in the value of the prognosis parameter.

10. The method of claim 1, further comprising monitoring a change in the value of the prognosis parameter over time to determine a trend in outcome of treatment delivered to patients in the institution having common diagnoses.

11. The method of claim 10, further comprising analyzing the trend to determine if a change in best practices for treating a condition is necessary.

12. The method of claim 10, further comprising analyzing the trend to determine if a change in level of care for treating a condition is necessary.

13. A method for determining a value for a harm index in real time, comprising:
obtaining current treatment related information about a patient;
identifying appropriate predictor variables;
inputting the treatment related information and the predictor variables into a prediction model;
calculating a value for a harm index.

14. The method of claim 13, wherein the prediction model is a logistic regression model.

15. The method of claim 13, wherein calculating a value for a harm index includes using calculated coefficients related to the predictor variables.

16. The method of claim 15, wherein the calculated coefficients are determined by analyzing a database of information containing treatment related information obtained from a plurality of patients.
